(19) United States
(12) Patent Application Publication
(10) Pub. No.: US 2017/0261446 A1
(43) Pub. Date: Sep. 14, 2017

(54) Method for Determining the Stability of a Petroleum Product Containing Asphaltenes

(71) Applicant: Total Raffinage Chimie, Courbevoie (FR)

(72) Inventors: Matthieu Adam-Berret, Thimeon (BE); Christian Lamotte, Arquennes (BE); Charles Marchal, Manage (BE); Maxime Lacroix, Uccle (BE); Gloria Vendrell, Le Havre (FR)

(21) Appl. No.: 15/503,661

(22) PCT Filed: Aug. 13, 2015
(86) PCT No.: PCT/EP2015/068666

§ 371 Date: Feb. 13, 2017

(30) Foreign Application Priority Data
Aug. 14, 2014 (FR) 1457806

(51) Int. Cl. G01N 24/08; C10G 9/00; C10G 11/18; G01R 33/44; G01N 33/28

(52) U.S. Cl. CPC G01N 24/085; G01R 33/448; G01N 33/2835; C10G 11/18; C10G 9/007; C10G 2300/206; C10G 2300/107; C10G 2300/1077; C10G 2300/1074; C10G 2400/02

(57) ABSTRACT

The invention relates to a method for determining a parameter representative of the stability of an asphaltene-containing petroleum product, said petroleum product being an effluent derived from a hydrocarbon feedstock conversion process or being a mixture of hydrocarbons, using proton NMR to determine a threshold value of said parameter representative of the stability, this threshold value constituting a boundary between a stability domain and an instability domain of a petroleum product. According to the invention, the parameter representative of the stability is a T₂ₘₑₐₙ/T₁ₘₑₐₙ or T₁ₘₑₐₙ/T₂ₘₑₐₙ ratio. The invention also relates to a method for monitoring a conversion process, in particular a deep conversion process, or a mixture of hydrocarbons, using this method of determination.

### FIELD OF THE INVENTION

The present invention relates to a method for determining the stability of an asphaltene-containing petroleum product, in particular containing hydrocarbons derived from the distillation of petroleum or else from a process for the deep conversion of petroleum.

The present invention also relates to a deep conversion process or to a process for formulating heavy fuel oils using said method.

### BACKGROUND OF THE INVENTION

Heavy petroleum products, and particularly petroleum distillation residues, effluents derived from thermal conversion processes, catalytic cracking processes, hydrocracking processes, deep hydroconversion processes, atmospheric or vacuum residue hydrotreating processes (ARDS or VRDS) or else the fuel oils derived from mixtures of heavy products, are mixtures of heavy hydrocarbons having a boiling point greater than or equal to 350° C., denoted by 350° C.⁺. They are mixtures of complex hydrocarbons, comprising colloidal systems constituted of asphaltenes.

Asphaltenes constitute the heaviest fractions of petroleum. They are shiny black solids, the molecular mass of which may vary from 1000 to 100 000 Da. Asphaltenes are concentrated in heteroelements and in metals: sulfur, nitrogen, nickel and vanadium. Asphaltenes may be defined as the family of molecules insoluble in n-pentane (C5) or n-heptane (C7).

Asphaltenes are thus highly aromatic heavy molecules having paraffinic side chains, heteroatoms such as S and N and that are dispersed (or else “peptized”) in the form of micelles in a heavy oil or organic phase. Asphaltenes have a great influence on the physicochemical properties of the heavy products. Specifically, asphaltenes have a capacity to flocculate and to be adsorbed on surfaces and to form solid deposits. These colloidal systems may be destabilized more or less easily, for example by thermal cracking, by the severity of the processes or by dilution.

The flocculation and precipitation phenomena are the cause of many problems both from the point of view of exploitation of deposits and of refining in deep conversion processes, during the storage of the effluents or the mixing of said effluents. Asphaltenes are precursors of coke, deactivate refining catalysts and impair the operation of refining equipment.

The determination of the flocculation threshold is an essential parameter for characterizing and predicting the stability of a heavy product.

In the field of refining petroleum, the asphaltene-containing petroleum products may be:
atmospheric residues or vacuum residues derived from the distillation of crude petroleum that contain from 2 to 25 wt % of asphaltenes;
effluents derived from thermal conversion processes, such as the visbreaking process, which contain from 10 to 30 wt % of asphaltenes;
effluents derived from catalytic cracking processes, such as the FCC (Fluid Catalytic Cracking) process, and of which the slurry cut (350° C.⁺ cut) contains from 0.1% to 8% by weight of asphaltenes;
effluents derived from hydrotreating processes, hydrocracking processes, (fixed-bed, moving-bed, ebullated-bed, entrained-bed or slurry-phase reactor (the catalyst of which is in suspension)) deep hydroconversion processes, or from the ARDS (Atmospheric Residue DeSulfurization) or VRDS (Vacuum Residue DeSulfurization) process, and which contain up to 50 wt % of asphaltenes;
pitches derived from physical separation processes, such as deasphalting, which contain from 4 to 50 wt % of asphaltenes;
mixtures of the petroleum products listed above for the formulation of heavy fuel oils that contain from 5 to 20 wt % of asphaltenes.

Each of the processes cited above will now be described in greater detail. The method(s) known and used to determine the stability of the effluent derived from said processes will also be described, as will the drawbacks linked to the use of said method(s).

Hydroconversion Processes

The feedstock consists of hydrocarbon-based feedstocks having an H/C ratio of at least 0.25. Thus, the hydrocarbon-based feedstocks that may be treated by these processes may be chosen from: atmospheric residues and vacuum residues, residues derived from a deasphalting unit, deasphalted oils, visbroken effluents (thermal cracking), 350° C.⁺ heavy effluents derived from an FCC unit, including the the FCC slurry (350° C.⁺) cut, schist oils, biomass, coal, petroleum coke from a delayed coker, or mixtures of one or more of these products. Other raw materials may also be co-treated with the petroleum residues: tyres, polymers, road asphalts.

These processes are characterized by harsh operating conditions: temperatures from 400° C. to 500° C., partial pressure of hydrogen from 90 to 250 bar and a large amount of catalyst (100 to 300 tonnes to treat 100 t/h of feedstock).

These processes may in particular be carried out with a fixed-bed or ebullated-bed catalyst described below.

Fixed-Bed Processes

Fixed-bed processes typically consist of a series of adiabatic reactors containing several catalyst beds. In each reactor, the gas and the liquid flow co-currently from top to bottom as a tri phase mixture, with the gas phase as the continuous phase. Mention may particular be made of the HYVAHL© process.

ARDS/VRDS processes are atmospheric or vacuum residue desulfurization processes. They make it possible to upgrade the atmospheric or vacuum residues and to eliminate undesirable contaminants and therefore to pretreat the feedstock for units located downstream in the refining layout such as the FCC. ARDS or VRDS processes customarily operate at temperature conditions between 350° C. and 450° C. (limits included) and preferably between 380° C. and 410° C. (limits included). The total pressure is in general from 90 to 200 bar, preferably from 150 to 170 bar.

The effluents at the outlet of an ARDS/VRDS unit, for example the 370° C.⁺ cuts, contain up to 5 wt % of asphaltenes.

Ebullated-Bed Processes

Ebullated-bed processes use a supported catalyst that is placed in suspension in the feedstock to be converted. The reactor is a column without internals where gas flows from bottom to top, which makes it possible to keep the catalyst in suspension. A fraction of the catalyst in the reactor is discharged and replaced by fresh catalyst. An ebullated-bed process enables operation at constant operating conditions with performance levels and a quality that are constant over time. Mention may be made of the commercial H-Oil© and LC-Fining© processes.

Slurry-Type Processes

The process in which the catalyst (or its precursor) is in powder form in suspension in the feedstock to be converted, subsequently referred to as “slurry-phase process” or slurry technology process, used for the hydroconversion of the heavy fractions of hydrocarbons is a process known to a person skilled in the art. The technologies of hydroconversion of residues in the slurry phase use a catalyst dispersed in the form of very small particles, the size of which is less than 500 μm, preferably from 1 to 200 nm, more particularly from 1 to 20 nm for liposoluble precursors. The catalysts, or their precursors, are injected with the feedstock to be converted at the inlet of the reactors. The catalysts pass through the reactors with the feedstocks and the products in the process of being converted, then they are entrained with the reaction products out of the reactors. They are recovered after separation in the heavy residual fraction that may contain from 0.05 (wt) % to 5 (wt) % of catalyst fines. The catalysts used as slurry are generally sulfide catalysts preferably containing at least one element chosen from the group formed by Mo, Fe, Ni, W, Co, V, Cr and/or Ru; these elements may be coupled in order to form bimetallic catalysts. In this type of process, the catalysts used are generally unsupported catalysts, that is to say that the active phase is not deposited on the surface of a porous solid support but is well dispersed directly in the feedstock. The catalyst is generally provided in a non-active form, referred to as precursor. The sulfurization of the catalytic metal present in the precursor makes it possible to obtain the metal sulfides that form the active phase of the catalysts. The precursors are generally conventional chemicals (metal salt, phosphomolybdic acid, sulfur-containing compounds, organometallic compounds or natural ores), which are converted into active catalyst in situ in the reactor or else in ex situ pre-treatment units that are an integral part of slurry-phase hydroconversion processes. The precursors are for example octoates, naphthenates, metallocenes, oxides or ground ores.

The catalyst may be used in a single pass or in recycle mode.

When the catalyst is in a non-active form, that is to say in the form of a precursor, it may be in liposoluble, water-soluble or (mineral) solid form, which are widely described in the literature.

Slurry-phase processes may operate according to various configurations. In one-pass mode, the catalyst at the outlet of the reactor is not recycled in the feedstock to be converted. The recycle mode is used when the catalyst retains an activity at the end of a first pass through the reactor. In recycle mode, the catalyst is concentrated after the reaction section and reinjected into the feedstock to be converted.

Specified in table 1 below, by way of example, are the amounts of catalysts that may be added to the feedstock whether in “one pass” mode or in “recycle” mode.

TABLE 1

| | Mo | Fe |
|---|---|---|
| Fat-soluble | 50 to 6000 ppm | 1000 ppm to |
| | (by weight) | 1% (by weight) |
| Water-soluble | 300 to 6000 ppm | 1500 ppm to |
| | (by weight) | 2% (by weight) |
| (Mineral) solid | 300 to 6000 ppm | 0.5% to 2% |
| | (by weight) | (by weight) |

The slurry-phase hydroconversion process operates at high severity in order to be able to convert complex feedstocks.

The process customarily operates at temperature conditions between 400° C. and 500° C. (limits included) and preferably between 410° C. and 470° C. (limits included). The pressure of hydrogen is in general from 90 to 250 bar, preferably from 100 to 170 bar. The liquid hourly space velocity, expressed in h⁻¹, which corresponds to the ratio of the throughput of the feedstock to the reaction volume, is for example between 0.05 and 1.5 h⁻¹ (limits included). This process may be carried out in one or more reactors, in series or in parallel, which may be of various types, for example a bubble-column or isothermal reactor.

Such a slurry-phase hydroconversion process may comprise, after a step of hydroconversion in at least one reactor containing a catalyst as slurry containing at least one metal, a step of separating the hydroconversion effluent. This separation step comprises 3 substeps:
First substep: the effluent of the hydroconversion step is separated into a C6⁻ cut and a C6⁺ cut at high temperature, around 300° C., and high pressure, around 150 bar, for example in a distillation column. The C6³⁰ effluents derived from this first substep are referred to as TLP for Total Liquid Product. The asphaltene content of said effluents is from 7% to 20% by weight.
Second substep: the C6⁺ cut separated in the previous step is separated into a 350° C.⁻ cut and a 350° C.⁺ cut at atmospheric pressure and at high temperature, around 300° C., for example in a distillation column. The asphaltene content of 350° C.⁺ cut is from 10% to 20% by weight.
Third substep: the 350° C.⁺ cut separated in the previous step is separated into a 525° C.⁻ cut and a 525° C.⁺ cut by vacuum distillation at high temperature, for example above 300° C. The 525° C.⁺ cut corresponds to the “final slurry” residue. Said residue consists of very complex molecules. A standard elemental composition of a final slurry residue is the following:

carbon: 84-87% (by weight)

hydrogen: 7-14% (by weight)

heteroelements: sulfur from 2 to 6 (wt) %, nitrogen from 0.5 to 2 (wt) %

metals: nickel and vanadium: 40 to 2000 ppm (by weight)

asphaltenes: 15-50 (wt) %

and optionally other elements in trace amounts.

The majority of the molecules have groups of aromatic rings optionally connected by paraffinic chains. They may contain more than 60% of carbon in unsaturated chains. The H/C atomic ratio is therefore low.

The severity of the operating conditions of these deep hydroconversion processes lies at the limit of the stability of the asphaltenes: as soon as the asphaltenes become unstable, they precipitate.

A person skilled in the art knows how to determine the stability of hydrocarbons derived from petroleum refining, and in particular of effluents including residues derived from deep hydroconversion processes, via various techniques that nevertheless have certain drawbacks:
Optical techniques by light dispersion, light absorption, light refraction. These techniques are not however predictive.
Acoustic techniques by ultrasonic attenuation. This technique has the drawback of being non-predictive and complex to implement industrially.
Electrical techniques by detecting the precipitation of asphaltenes, which are not predictive and the use of which on heavy products is not suitable.
Techniques for determining the precipitation threshold by addition of a paraffinic solvent described for example in the ASTM D7060, ASTM D7112 or else ASTM D7157 standards and for which the detection of the precipitation threshold is not precise.
Magnetic technique by NMR (Nuclear Magnetic Resonance) as developed by the Institut Francais du Pétrole [French Institute of Petroleum] and for which a patent FR 2 834 792 has been filed. This technique consists in detecting and in monitoring the kinetics of flocculation of the high molecular weight fractions of a complex fluid by nuclear magnetic resonance. This technique has the drawback of being particularly long and complex due to the use of high-resolution NMR.

Regarding the processes that will be described below, the stability of the asphaltenes is determined by measuring the S-value according to ASTM D7157.

FCC (Fluid Catalytic Cracking) Processes

The feedstocks treated are distillates obtained by vacuum distillation, visbreaker distillates and also residues as long as the metal content thereof is acceptable.

The process customarily takes place under temperature conditions from 480° C. to 540° C. and pressure conditions from 2 to 3 bar with a specific cracking catalyst.

The unit produces heavy gasolines (160° C.-220° C.), an LCO cut (220° C.-350° C.) and a slurry cut (350° C.⁺).

The slurry cut contains from 0.1 to 8 wt % of asphaltenes.

Visbreaking Process

This process is applied to atmospheric residues and vacuum residues that contain from 2 to 25 wt % of asphaltenes.

The temperatures at the outlet of the furnace are from 400° C. to 490° C. depending on the feedstock to be treated. The unit may also comprise a maturing chamber where the reaction continues. The pressure is from 5 to 12 bar. The severity of the cracking should be controlled to avoid obtaining an unstable heavy fuel oil.

The heaviest fractions derived from the visbreaking process are the atmospheric and vacuum residue (350° C.⁺-500° C.). This is a fuel oil with improved viscosity with respect to the feedstock. The visbroken residue (VBR) may contain from 15 to 30 wt % of asphaltenes.

The modification of the structure of the asphaltenes during the cracking process and also the change in characteristics of the oily medium risk leading to a destabilization of the colloidal solution and giving rise to the precipitation of the asphaltenes hence the need to have available a rapid and effective method for determining the stability level of the product.

A person skilled in the art knows how to measure the stability of the asphaltene-containing effluents, including residues, derived from visbreaking processes, catalytic cracking (FCC) processes or deep hydroconversion processes, in particular by techniques for determining the precipitation threshold by addition of a solvent and, for example, the standardized method for measuring the S-value (ASTM D7157). This is a method for estimating the intrinsic stability of a heavy product according to the formula:

$$S = \frac{{S}_{0}}{1 - {S}_{a}}$$

with Sa: stability of the asphaltenes and So: solvency of the oily medium. However, the method for determining the S-value has several drawbacks:
preparation of the samples which requires the use of solvents
carrying out the measurements in a laboratory
duration of the analysis
a posteriori analysis of the effluent which does not make it possible to correct the implementation of the process in real time.

Heavy Fuel Oils

Fuel oils are derived from mixtures of two families of constituents: heavy bases and fluxes.

The heavy base consists for example of vacuum residues derived from the visbreaking process, or else of atmospheric or vacuum residues derived from the distillation of crude petroleum.

The flux consists for example of the slurry cut derived from the FCC process, of Light Cycle Oil (LCO), of Heavy Cycle Oil (HCO), of kerosene and/or of gas oil derived from the distillation of crude petroleum, of gas oil (165° C.-350° C. cut) derived from the visbreaking process, of heavy gasoline (160° C.-220° C. cut) derived from the FCC process, of pyrolysis oil derived from the naphtha steam cracking process.

The asphaltene content of the mixtures forming the heavy fuel oils is between 5 and 20 wt %.

One of the greatest problems in the use of heavy fuel oils concerns the risks of incompatibility in the operations of mixing products of different provenance. This results in a more or less rapid precipitation of the asphaltenes in the form of sludge which may clog the pipes and filters.

The stability of the fuel oils is determined by two measurements: the measurement of the S-value as described above and the measurement of the sediment content according to the ISO10307-2 standard or according to the ISO 10307-1 standard. This measurement determines the propensity of the asphaltenes to precipitate but nevertheless has the following drawbacks:
The sample to be analyzed must be heated for 24 h then filtered. The ability to act on the composition of the mixture and to correct this composition is therefore poor when at the limit of the stability of said mixture.
The detection of the fuel oils at the limit of the stability is difficult.

There is therefore a need for petroleum products, derived from the processes described above or forming heavy fuel oils, and that contain asphaltenes, to have available a rapid and reliable method for determining the stability thereof.

### DESCRIPTION OF THE INVENTION

A rapid and reliable method for determining a parameter representative of the stability of an asphaltene-containing petroleum product using proton NMR is thus proposed.

Specifically, the applicant has found that the use of proton NMR, and in particular low-field NMR, in the petroleum refining field, and in particular applied to asphaltene-containing petroleum products, made it possible to carry out a monitoring of the stability of said petroleum products and to predict the risks of precipitation.

Furthermore, this method of characterizing the stability of asphaltene-containing petroleum products uses a parameter T₂ₘₑₐₙ/T₁ₘₑₐₙ or T₁ₘₑₐₙ/T₂ₘₑₐₙ determined by proton NMR. Furthermore, the T₂ₘₑₐₙ/T₁ₘₑₐₙ or T₁ₘₑₐₙ/T₂ₘₑₐₙ parameter may be determined by low-field proton NMR, also referred to as time-domain NMR.

In the latter case, the advantages identified are the following:
method that does not require sample preparation in order to determine the stability of the effluent and therefore the use of solvent and an optional filtration step are avoided;
rapidity of the measurement;
predictive implementation of a deep conversion process, anticipating the risks of destabilization of the effluent.

It is known to use low-field NMR in the petroleum field, for example in the field of crude petroleum exploration:
assaying the percentage of hydrogen in a petroleum cut;
assaying the oil content in the paraffins;
characterizing wellbores in order to determine the presence and the amount of extractable oil and also the wettability;
in situ determination of the viscosity of samples of crude petroleum that was the subject of patent application CA 2 638 697 by Schlumberger filed in 2008.

The use of low-field NMR is also known for fields as varied as the agri-food industry or the formulation of construction products. These various uses have been the subject of a publication entitled “Low-field permanent magnets for industrial process and quality control” published in Progress in Nuclear Magnetic Resonance Spectroscopy, volume 76 in January 2014, pages 1-60.

Aside from the document FR 2 834 792 already mentioned, proton NMR, especially low-field proton NMR, has not however been used until now for monitoring the stability of asphaltene-containing hydrocarbons.

In order to facilitate understanding, the following terms will be defined:

Stability: The conversion of heavy petroleum products to lighter products is limited by the content of asphaltenes. At high concentration, namely for a concentration greater than or equal to 0.1% by weight, since the residual asphaltenes are no longer soluble in the medium, there is a risk of precipitation of these asphaltenes which leads to the production of unstable residues that are unsuitable for sale. The same risk of precipitation exists during the production of mixtures of asphaltene-containing hydrocarbons, in particular for manufacturing heavy fuel oils. In other words, a petroleum product is considered to be stable in the absence of precipitation of the asphaltenes and unstable when the asphaltenes precipitate.

It should be noted that in the present description the terms “flocculation” and “precipitation” are used interchangeably.

The stability of a petroleum product may thus be defined by the suspended state of the asphaltenes that are present in said petroleum product. When the asphaltenes are in suspension, it is then said that the asphaltenes are peptized in the carbon-based matrix or else are metastable. In this state of stability, the asphaltenes remain dispersed within said matrix, as opposed to an unstable petroleum product where the asphaltenes precipitate, in particular on the walls of the support containing said petroleum product.

It is generally acknowledged that the attractive intermolecular interactions between the asphaltenes are the cause of their aggregation and of the instability of a petroleum product. The chemical reactions which take place during a refining thermal conversion process strengthen the attractive interaction forces between the asphaltenes. The instability of the effluents is demonstrated by solid deposits of clusters of asphaltenes that form on supports such as pipelines, pumps, reactors and production equipment. These deposits then cause pressure drops, fouling or even plugging of units/lines, blockages of pumps, and may lead to the shutdown of the unit.

Low-field NMR or time-domain NMR: This is a technique that is based on the relaxation time of the protons (¹H) and makes it possible to characterize the system by the differences in mobility of the molecules. The sample to be analyzed is introduced into a constant magnetic field B₀ and its response is studied after a specific excitation (pulse B₁). It is referred to as low-field NMR when the magnetic fields B₀ involved vary from 10 mT to 1.4 T approximately, i.e. for the ¹H proton, Larmor frequencies v₀ ranging from 425 KHz to 60 MHz.

When the nuclei, that is to say the ¹H protons, are excited by one or more magnetic field pulses B₁, they return to their equilibrium state according to several relaxation mechanisms. These relaxation phenomena are directly linked to the mobility of the molecules and provide information on their environment. It is possible to characterize these phenomena mainly by two relaxation times:
The longitudinal relaxation or spin-lattice relaxation (T₁), which corresponds to the return to equilibrium of the longitudinal magnetization (i.e. magnetization parallel to the magnetic field B₀), denoted by Mz.

The relaxation signals T₁ are adjusted by means of the following equation:

$$Mz ( t ) = {\sum }_{i}^{n} {A}_{i} \times [ 1 - ∝ \times exp ( - \frac{t}{{T}_{1 i}} ) ] \qquad ( 1 )$$

With Mz (t): magnetization along the axis of B₀ as a function of the time t

Aᵢ: weight fraction

T₁ᵢ: longitudinal relaxation time of the fraction i of the sample

i and n: non-zero integers, i varying from 1 to n, n being the minimum number of components that can be used to adjust the signal.

With α=1 or 2 depending on the NMR sequence used. If the sequence used is an inversion-recovery sequence then α=2. If the sequence used is a saturation-recovery sequence then α=1.

Other equations representative of the adjustment of T₁ may also be used.

It is possible to then calculate T₁ₘₑₐₙ which corresponds to the arithmetic mean.

$${T}_{1 mean} = \frac{{\sum }_{i} {A}_{i} {T}_{i 1}}{{\sum }_{i} {A}_{i}} \qquad ( 2 )$$

i: non-zero integer

Aᵢ: weight fraction i of the sample,

T₁ᵢ: longitudinal relaxation time of the fraction i of the sample.

As a variant, T₁ₘₑₐₙ may also be the result of the geometric or logarithmic mean.
The transverse relaxation or spin-spin relaxation (T₂), which corresponds to the return to equilibrium of the transverse magnetization (i.e. magnetization in the plane perpendicular to the magnetic field B₀), denoted by Mx.

The T₂ relaxation signals of the hydrocarbons containing a rigid phase and a mobile phase are adjusted by means of the following equation:

$${M}_{x} ( t ) = {A}_{1} \times exp ( - \frac{{t}^{2}}{{T}_{21}^{2}} ) + {\sum }_{i = 2}^{n} [ {A}_{i} \times exp - \frac{t}{{T}_{2 i}} ] \qquad ( 3 )$$

Mx(t): magnetization along the axis perpendicular to B₀ as a function of the time t.

A₁: weight fraction 1 of the sample

Aᵢ: weight fraction i of the sample

T₂₁: transverse relaxation time of the fraction 1 of the sample

T₂ᵢ: transverse relaxation time of the fraction i of the sample

i and n: non-zero integers, i varying from 2 to n, n being the minimum number of components that can be used to adjust the signal.

In order to obtain a good adjustment of the signal, n will be chosen with the smallest possible sufficient value, for example from 3 to 5.

Other equations representative of the adjustment of T₂ may also be used.

It is possible to then calculate T₂ₘₑₐₙ which corresponds to the arithmetic mean.

$${T}_{2 mean} = \frac{{\sum }_{i} {A}_{i} {T}_{2 i}}{{\sum }_{i} {A}_{i}} \qquad ( 4 )$$

i: non-zero integer varying from 1 to n

Aᵢ: weight fraction i of the sample,

T₂₁: transverse relaxation time of the fraction i of the sample.

As a variant, T₂ₘₑₐₙ may also be the result of the geometric or logarithmic mean.

Several NMR sequences may be used to measure the relaxation times T₁ and T₂ of petroleum products. These are chosen from the sequences known to a person skilled in the art and which make it possible to measure the relaxation times of all of the phases of the sample (rigid phases and mobile phases). Within the context of this invention, the NMR sequences below may be used:

FID (Free Induction Decay), which enables the measurement of the decay of the signal of the rigid phases. It can only be used over the time range 0-200 is, since above 200 is it is too sensitive to the inhomogeneity of the magnetic field B₀.

Inversion-recovery (T1 IR) or saturation-recovery (T1 SR) sequences that make it possible to characterize the T₁ value(s) of the sample.

The FID-CPMG (Carr-Purcell-Meiboom-Gill) sequence which makes it possible to characterize T₂. This sequence is a novel sequence that combines the measurement of the FID over 75 μs, then a measurement of the longer T₂ value(s) without being affected by the inhomogeneity of the magnetic field B₀.

The measurements of the relaxation times T₁ and T₂ may be carried out one after the other in any order or else using sequences that enable the simultaneous measurement of T₁ and T₂.

From a physical point of view, T₁ is always greater than or equal to T₂. For an unconfined pure liquid phase, T₁=T₂.

### DETAILED DESCRIPTION OF THE INVENTION

Other advantages and features will emerge more clearly from the description which will follow and the particular embodiments of the invention of which are given as nonlimiting examples.

The present invention consists in proposing a method for determining a parameter representative of the stability of an asphaltene-containing petroleum product, said petroleum product being an effluent derived from a hydrocarbon feedstock conversion process or being a mixture of hydrocarbons. In particular, as already mentioned, a precipitation of the asphaltenes results in an instability of said petroleum product.

According to the invention, the method comprises the following steps:
a. various petroleum products, in particular constituting various samples of said petroleum product, are prepared either by implementing said process for the conversion of one and the same hydrocarbon feedstock at various conversion levels, or by mixing hydrocarbons in different proportions,
b. the longitudinal relaxation times T₁ and the transverse relaxation times T₂ of the various petroleum products prepared in step a) are measured by proton NMR,
c. as parameter representative of the stability, a T₂ₘₑₐₙ/T₁ₘₑₐₙ or T₁ₘₑₐₙ/T₂ₘₑₐₙ ratio is determined for each of the petroleum products prepared in step a), wherein T₂ₘₑₐₙ and T₁ₘₑₐₙ are the means of the values measured during step b),
d. a threshold value of said parameter representative of the stability is determined, constituting a boundary between a stability domain and an instability domain of a petroleum product

Thus, in use, the value of the T₂ₘₑₐₙ/T₁ₘₑₐₙ ratio (or of its reciprocal T₁ₘₑₐₙ/T₂ₘₑₐₙ) of a product may be monitored and compared to the threshold value. Optionally, a function of this T₂ₘₑₐₙ/T₁ₘₑₐₙ or T₁ₘₑₐₙ/T₂ₘₑₐₙ ratio may be used to carry out the stability monitoring of the petroleum product.

For a predictive purpose, it is possible to consider a monitoring threshold corresponding to the threshold value ±10%.

According to one variant, in the course of step c), the ratio T₂ₘₑₐₙ/T₁ₘₑₐₙ can be determined as parameter representative of the stability.

In this case, this value is such that a stable petroleum product has a T₂ₘₑₐₙ/T₁ₘₑₐₙ ratio lower than said threshold value, an unstable petroleum product having a T₂ₘₑₐₙ/T₁ₘₑₐₙ ratio above said threshold value. Depending on the application, the threshold value may or may not be included in the stability domain.

For a predictive purpose, it is then possible to consider that the petroleum product is stable if its T₂ₘₑₐₙ/T₁ₘₑₐₙ ratio is strictly lower than the threshold value. For greater safety, a product may be considered to be stable if its T₂ₘₑₐₙ/T₁ₘₑₐₙ ratio is a predetermined percentage lower than the threshold value, for example 10% lower.

According to another variant, the reciprocal T₁ₘₑₐₙ/T₂ₘₑₐₙ ratio may be determined during step c).

Advantageously, when the petroleum product is an effluent derived from a conversion process, this conversion process may be chosen from a thermal conversion process, a fluid catalytic cracking process, a hydrocracking process, a hydrotreating process, a fixed-bed hydroconversion process, a moving-bed hydroconversion process, an ebullated-bed hydroconversion process, a slurry-phase hydroconversion process, a vacuum distillation residue desulfurization process or an atmospheric distillation residue desulfurization process.

The petroleum product derived from the conversion process may be an effluent of said process or maybe a cut of the effluent of said process. In particular, the petroleum product may be a heavy petroleum product, as defined previously.

The conversion is said to be deep when the process makes it possible to recover more light molecules. Then, at the end, there remains only a minimal amount of very heavy products, such as very heavy fuel oils and coke.

Advantageously, when the petroleum product is a mixture of hydrocarbons, it is a heavy fuel oil, as defined above.

Advantageously, said petroleum products have an asphaltene content of at most 50% by weight and of at least 0.1% by weight.

The asphaltene content may be at least 1% by weight, or even at least 2% by weight.

As a variant or in combination, the asphaltene content may be at most 30%, or even at most 25% or at most 20%.

The method according to the invention may be applied to petroleum products having asphaltene contents of from 0.1% to 8% by weight, from 1% to 5% by weight, from 2% to 25% by weight, from 5% to 20% by weight, from 7% to 20% by weight, from 10% to 20% by weight, from 10% to 30% by weight, from 15% to 30% by weight, from 4% to 50% by weight or from 15% to 50% by weight.

A person skilled in the art will easily be able to determine the number of samples of petroleum product prepared in step a), in particular a sufficient number to determine a stability threshold. For example, in the case where the samples are obtained by implementing the process for conversion of one and the same feedstock at various conversion levels, these conversion levels may be close to the most commonly used conversion levels and/or flank these conversion levels or conversion levels envisaged for the process in question, or else flank a conversion level for which an instability is often observed. Similarly, when the petroleum product is a mixture, the proportions of the samples may be close to the proportions customarily used or envisaged, and/or flank these proportions or else the proportions of a mixture for which an instability is often observed. Generally, from 2 to several tens of samples, preferably from 2 to 50 samples, preferentially from 2 to 30 samples, for example from 4 to 15 samples, may be provided.

According to one particular embodiment, the method according to the invention may also comprise, during step d) of determining a threshold value, a step of determining S-values of each of the petroleum products prepared in step a) according to the ASTM D7157 method, said threshold value being chosen equal to the T₂ₘₑₐₙ/T₁ₘₑₐₙ or T₁ₘₑₐₙ/T₂ₘₑₐₙ mean ratio of the mixture whose S-value indicates the stability threshold of the asphaltenes.

Optionally, for greater safety, this threshold value may be chosen so as to be a predetermined percentage lower (respectively higher) than the value of the T₂ₘₑₐₙ/T₁ₘₑₐₙ (respectively T₁ₘₑₐₙ/T₂ₘₑₐₙ) ratio of the mixture whose S-value indicates the stability threshold of the asphaltenes. This percentage is for example 10%.

This embodiment is more particularly suitable for the petroleum products for which the S-value method can be used, for example heavy fuel oils.

According to another particular embodiment, when the T₂ₘₑₐₙ/T₁ₘₑₐₙ ratio is used as parameter, the method according to the invention may also comprise, during step d) of determining a threshold value, a step of plotting a curve of the T₂ₘₑₐₙ/T₁ₘₑₐₙ ratios calculated as a function of the composition of said petroleum products prepared in step a), said threshold value corresponding to a value of the T₂ₘₑₐₙ/T₁ₘₑₐₙ ratio for which a plateau is arrived at.

Such a plateau may be observed for petroleum product compositions that are considered to be unstable.

A plateau is understood to mean a zone of the curve in which the T₂ₘₑₐₙ/T₁ₘₑₐₙ ratio is constant or almost constant. A ratio is said to be almost constant when its value does not vary by more than 10%, in particular does not vary by more than 5%, as a function of the composition of the petroleum products.

In particular, the curve of the ratios may be plotted as a function of the conversion levels of each of the petroleum products prepared in step a) when these petroleum products are derived from a conversion process.

Optionally, as for the previous embodiment, the threshold value may be chosen so as to be a predetermined percentage lower than the value of the T₂ₘₑₐₙ/T₁ₘₑₐₙ ratio for which a plateau is arrived at, this percentage being for example 10%.

It will thus be noted that the threshold value may be determined using measurements of T1 and T2, as described previously, or by any other suitable method known to a person skilled in the art, such as the measurement of the S-value described above, or else by direct observation of the precipitation, by filtration, etc. Step d) of the present invention is thus not limited to one particular method of determining the threshold value.

Advantageously, for a petroleum product, in particular an unfiltered petroleum product, derived from a slurry-phase conversion process, it may be considered that if T₂ₘₑₐₙ/T₁ₘₑₐₙ is greater than or equal to 0.85 then the system is unstable and it is close to flocculation. In other words, the threshold value of the T₂ₘₑₐₙ/T₁ₘₑₐₙ ratio is then 0.85.

Advantageously, for a filtered petroleum product derived from a slurry-phase conversion process, it may be considered that if T₂ₘₑₐₙ/T₁ₘₑₐₙ is greater than or equal to 0.27 then the system is unstable and it is close to flocculation. In other words, the threshold value of the T₂ₘₑₐₙ/T₁ₘₑₐₙ ratio is then 0.27.

The main objective of the filtration is in particular to eliminate the flocculated asphaltenes. Thus, any suitable method for eliminating these flocculated asphaltenes may be used.

According to the invention, the probe used in step b) advantageously has a dead time of less than or equal to 11 ps.

According to the invention, it is preferably a question of proton NMR measurements, in particular low-field proton NMR measurements.

The invention also relates to a method for monitoring a conversion process, in particular a deep conversion process, comprising the following steps:
a. a heavy hydrocarbon-based feedstock having an H/C ratio of at least 0.25 is converted,
b. the effluents produced by said conversion are recovered and at least one predetermined cut is separated,
c. the T₂ₘₑₐₙ/T₁ₘₑₐₙ or T₁ₘₑₐₙ/T₂ₘₑₐₙ ratio of said cut is measured by NMR,
d. the T₂ₘₑₐₙ/T₁ₘₑₐₙ or T₁ₘₑₐₙ/T₂ₘₑₐₙ ratio determined in step c) is compared to a threshold value of the parameter representative of the stability previously determined by means of the method of determination according to the features defined previously using various conversion levels of said feedstock,
e. it is deduced therefrom whether or not the predetermined cut is stable.

The invention finally relates to a method for monitoring a mixture of hydrocarbons, in particular for manufacturing a heavy fuel oil, wherein, during the mixing:
a. the T₂ₘₑₐₙ/T₁ₘₑₐₙ or T₁ₘₑₐₙ/T₂ₘₑₐₙ ratio of said mixture is measured by NMR,
b. the T₂ₘₑₐₙ/T₁ₘₑₐₙ or T₁ₘₑₐₙ/T₂ₘₑₐₙ ratio determined in step a) is compared to a threshold value of the parameter representative of the stability previously determined by means of the method of determination according to the invention using mixtures of the same hydrocarbons in different proportions,
c. it is deduced therefrom whether or not the mixture is stable.

In one or other of these monitoring methods, it is thus possible, in the course of the process or mixing, to adapt the operating conditions in order to remain within the stability domain of the product in question.

The comparison steps d) and b) of these monitoring methods may consist of a simple comparison between the ratio considered and the threshold value or may consist of a monitoring of a function of the ratio considered (logarithmic function, exponential function, linear combination function, etc.) with respect to the value of this function for the threshold value.

These steps of comparing and of determining the stability may be carried out by a management system such as a processor of microprocessor, microcontroller or other type, for example a CPU (Central Processing Unit). The data measured may be stored in storage means which may be a RAM (Random Access Memory), an EEPROM (Electrically-Erasable Programmable Read-Only Memory), or other.

The management system may be part of the management system controlling the process or the mixing.

### FIGURES

FIG. 1 is a graphical representation of the T₂ₘₑₐₙ/T₁ₘₑₐₙ ratio as a function of the conversion level (example A).

FIG. 2 is a graphical representation of the T₂ₘₑₐₙ/T₁ₘₑₐₙ ratio as a function of the value of Sa (example B).

### EXAMPLES

The examples below aim to illustrate the effects of the invention and its advantages, but without limiting the scope thereof.

### Example A

### With a 250° C.⁺ TLP Derived from a Slurry-Phase Hydroconversion Process

The estimation of the stability of a 250° C.⁺ effluent (TLP) derived from a deep conversion process in a slurry reactor will be carried out as follows.

Preparation of the samples: approximately 1 ml of the effluent/feedstock to be analyzed by NMR is withdrawn and poured into the bottom of an NMR tube. Since the feedstock and the effluents are very viscous at ambient temperature, it is necessary to heat the sample by passing into an oven at 110° C. for at least 5 min, in order to homogenize it and to liquefy it in order to be able to withdraw it.

The measurements were carried out using a 0.47 T Bruker Minispec MQ20 spectrometer operating at 20 MHz for the proton, equipped with a 10 mm probe and having a dead time of 7 ps. The term “dead time” is understood to mean the time starting from which it is possible to record the signal. The mean duration of the 90° and 180° pulses is respectively 2.6 ps and 5.3 ps.

Step a: The longitudinal relaxation time T₁ is measured at 60° C. on various samples. In order to do this, a sample of the 250° C.+TLP (Total Liquid Product) is withdrawn at various conversion levels.
Sample no. 1 corresponds to the feedstock derived from Safaniya crude oil, the asphaltene content of which is from 7% to 20% by weight. The asphaltenes are completely within the TLP samples used below
Sample no. 2 corresponds to the effluent (TLP) at a conversion level of 22%
Sample no. 3 corresponds to the effluent at a conversion level of 51%
Sample no. 4 corresponds to the effluent at a conversion level of 73%
Sample no. 5 corresponds to the effluent at a conversion level of 80.5%
Sample no. 6 corresponds to the effluent at a conversion level of 92%.

The T₁ values are measured for the above samples and the weighted mean of these T₁ values is produced for each of the samples as a function of the representation in % of the conversion levels of the sample using equation (2). These values are collated in table 2.

Step b: The transverse relaxation time T₂ of the samples is measured by low-field NMR and T₂ₘₑₐₙ is calculated by weighting of the T₂ as a function of the representation in % of the conversion levels of the sample using equation (4). These values are collated in table 2.

Step

A T₂ₘₑₐₙ/T₁ₘₑₐₙ ratio is determined, the values of which are presented in table 2.

TABLE 2

| | % | | | |
|---|---|---|---|---|
| | CONVERSION | | | T₂ₘₑₐₙ/T₁ₘₑₐₙ |
| Sample | 525° C.⁺ | T_{1 mean} (ms) | T_{2 mean} (ms) | (within ± 5%) |
| No. 1 | 0 | 78 | 0.2 | 0.003 |
| No. 2 | 22% | 57 | 2.2 | 0.04 |
| No. 3 | 51% | 87 | 25.0 | 0.29 |
| No. 4 | 73% | 192 | 156 | 0.81 |
| No. 5 | 81% | 497 | 481 | 0.97 |
| No. 6 | 92% | 786 | 787 | 1 |

The conversion level (or conversion) may be defined as being the ratio:
$\frac{\begin{matrix}wt \% of the 525 ° C {.}^{+} cut in the feedstock - \\ wt \% of 525 ° C {.}^{+} cut in the effluents\end{matrix}}{wt \% of the 525 ° C {.}^{+} cut in the feedstock}$

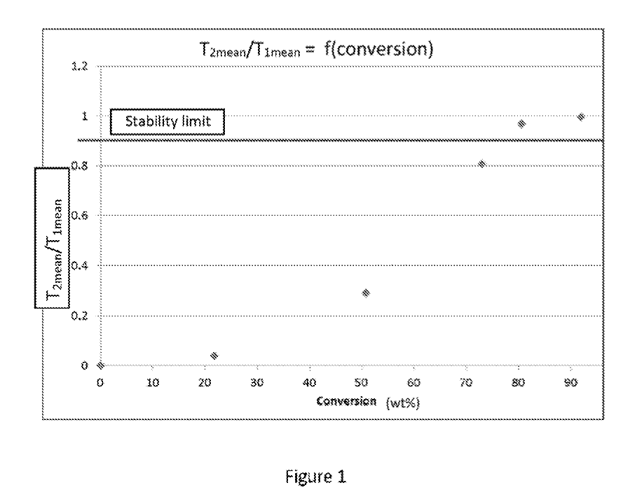

A graphical representation of the T₂ₘₑₐₙ/T₁ₘₑₐₙ ratio is plotted as a function of the conversion level, as represented in FIG. 1.

On this graph, it is seen that with the method in accordance with the invention, it is possible to determine the flocculation threshold of an asphaltene-containing petroleum product. This graphical determination is confirmed by a visual observation of the samples according to whether or not this product is precipitated.

For an unfiltered effluent derived from the slurry-phase conversion process, it may be considered that if T₂ₘₑₐₙ/T₁ₘₑₐₙ is greater than 0.85 then the system is unstable and there is a risk of flocculation.

For a predictive purpose, it is possible to consider that the T₂ₘₑₐₙ/T₁ₘₑₐₙ ratio may be up to 10% lower than said threshold value.

### Example B

### With a Filtered 250° C.⁺ TLP Derived from a Slurry-Phase Hydroconversion Process

The estimation of the stability of a filtered 250° C.⁺ effluent (TLP) derived from a deep conversion process in a slurry reactor will be carried out as follows.

The preparation of the samples is in accordance with that used in the course of example A.

The feedstock used to produce TLPs contains from 10% to 20% by weight of asphaltenes. The asphaltenes are completely within the TLPs. After filtration, the content of asphaltenes in the filtered TLP is from 8% to 18% by weight.

The TLP contains both flocculated asphaltenes and asphaltenes that are at the flocculation limit. If the sample is filtered, the flocculated asphaltenes are removed from the sample and only non-flocculated, and therefore stable, asphaltenes remain.

In order to carry out filtration, the TLP is diluted in toluene. After dilution, a vacuum filtration is carried out. It is possible to use a glass fiber filter having a porosity of 0.7 μm. The residual toluene is evaporated by a rotary evaporator under nitrogen.

The measurement of the S-value, according to the ASTM D7157 standard, is carried out so as to obtain values of Sa, that is to stay the intrinsic stability of the asphaltenes.

The results are collated in table 3 below.

The value of T₂ₘₑₐₙ and T₁ₘₑₐₙ is determined in accordance with the method used during example A. These values are collated in table 3.

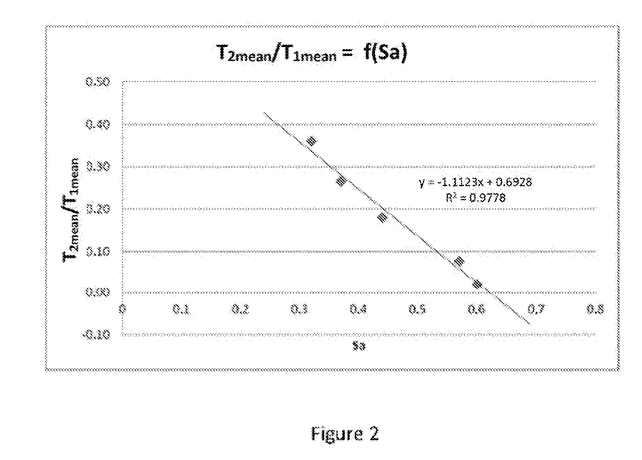

The results obtained enable a correlation to be obtained between the T₂ₘₑₐₙ/T₁ₘₑₐₙ ratio and the value of Sa, represented in FIG. 2.

It is known, for carrying out the process, that the stability threshold of the asphaltenes (Sa) determined by the S-value should be greater than 0.35.

The determination of the T₂ₘₑₐₙ/T₁ₘₑₐₙ ratio and the correlation with the value of Sa makes it possible to observe that T₂ₘₑₐₙ/T₁ₘₑₐₙ should be kept below 0.27 in order to keep the system stable.

TABLE 3

| | | | | | | | | T₂ₘₑₐₙ/ |
|---|---|---|---|---|---|---|---|---|
| | | | | | | | | T₁ₘₑₐₙ |
| Sample | Temperature | Conversion | | | | | | (within |
| no. | (° C.) | 525° C.⁺ (% m) | S | Sa | So | T₂ₘₑₐₙ | T₁ₘₑₐₙ | ± 5%) |
| 7 | 430 | 36% | 2.14 | 0.57 | 0.92 | 4.3 | 55.7 | 0.08 |
| 8 | 433 | 39% | 2.19 | 0.6 | 0.87 | 1.2 | 56 | 0.02 |
| 9 | 439 | 51% | 1.40 | 0.44 | 0.78 | 13.8 | 76.3 | 0.18 |
| 10 | 435 | 58% | 1.18 | 0.37 | 0.75 | 28.4 | 107 | 0.27 |
| 11 | 435 | 63% | 1.30 | 0.32 | 0.89 | 50.9 | 141.1 | 0.36 |

The conversion level (or conversion) may be defined as being the ratio:
$\frac{\begin{matrix}wt \% of the 525 ° C {.}^{+} cut in the feedstock - \\ wt \% of 525 ° C {.}^{+} cut in the effluents\end{matrix}}{wt \% of the 525 ° C {.}^{+} cut in the feedstock}$

1.-13. (canceled)

14. A method for determining a parameter representative of the stability of an asphaltene-containing petroleum product, a precipitation of which results in an instability of the petroleum product, the petroleum product being an effluent derived from a hydrocarbon feedstock conversion process or being a mixture of hydrocarbons, the method comprising:
a. preparing one or more petroleum products either by implementing the process for the conversion of one and the same hydrocarbon feedstock at a plurality of conversion levels, or by mixing hydrocarbons in different proportions,
b. measuring the longitudinal relaxation times T₁ and the transverse relaxation times T₂ of the petroleum products prepared in step a) by proton NMR,
c. determining as parameter representative of the stability, a T₂ₘₑₐₙ/T₁ₘₑₐₙ or T₁ₘₑₐₙ/T₂ₘₑₐₙ ratio for each of the petroleum products prepared in step a), wherein T₂ₘₑₐₙ and T₁ₘₑₐₙ are the means of the values measured during step b), and
d. determining a threshold value of the parameter representative of the stability, the threshold value constituting a boundary between a stability domain and an instability domain of the petroleum product.

15. The method according to claim 14, wherein step c) comprises determining the ratio T₂ₘₑₐₙ/T₁ₘₑₐₙ as a parameter representative of the stability.

16. The method according to claim 14, wherein the conversion process comprises a thermal conversion process, a fluid catalytic cracking process, a hydrocracking process, a hydrotreating process, a fixed-bed hydroconversion process, a moving-bed hydroconversion process, an ebullated-bed hydroconversion process, a slurry-phase hydroconversion process, a vacuum distillation residue desulfurization process or an atmospheric distillation residue desulfurization process.

17. The method according to claim 14, wherein the petroleum product is a heavy fuel oil.

18. The method according to claim 14, wherein the petroleum products have an asphaltene content of at most 50% by weight and of at least 0.1% by weight.

19. The method according to claim 14, wherein step d) of determining a threshold value comprises a step of determining S-values of each of the petroleum products prepared in step a), an S-value being defined by the formula:
$$S = \frac{{S}_{0}}{1 - {S}_{a}}$$
with Sa: stability of the asphaltenes and S₀: solvency of the oily medium, the threshold value being chosen equal to the T₂ₘₑₐₙ/T₁ₘₑₐₙ or T₁ₘₑₐₙ/T₂ₘₑₐₙ ratio of the petroleum product whose S-value indicates a precipitation of the asphaltenes.

20. The method according to claim 15, wherein step d) of determining a threshold value comprises a step of plotting a curve of the T₂ₘₑₐₙ/T₁ₘₑₐₙ ratios calculated as a function of the composition of the petroleum products prepared in step a), the threshold value corresponding to a value of the T₂ₘₑₐₙ/T₁ₘₑₐₙ ratio for which a plateau is arrived at.

21. The method according to claim 20, wherein, when a petroleum product that is an effluent derived from a slurry-phase conversion process has a T₂ₘₑₐₙ/T₁ₘₑₐₙ ratio greater than or equal to 0.85, then the petroleum product is close to flocculation.

22. The method according to claim 20, wherein, when a petroleum product derived from a slurry-phase conversion process and filtered has a T₂ₘₑₐₙ/T₁ₘₑₐₙ ratio greater than or equal to 0.27, then the mixture is close to flocculation.

23. The method according to claim 1, wherein, in step b), a probe having a dead time of less than or equal to 11 μs is used.

24. The method according to claim 1, wherein the NMR measurements are low-field proton NMR measurements.

25. A method for monitoring a a deep conversion process comprising:
a. converting a heavy hydrocarbon-based feedstock having an H/C ratio of at least 0.25,
b. recovering the effluents produced by the conversion and separating at least one predetermined cut,
c. measuring the T₂ₘₑₐₙ/T₁ₘₑₐₙ or T₁ₘₑₐₙ/T₂ₘₑₐₙ ratio of the predetermined cut by NMR,
d. comparing the T₂ₘₑₐₙ/T₁ₘₑₐₙ or T₁ₘₑₐₙ/T₂ₘₑₐₙ ratio determined in step c) to a threshold value of the parameter representative of the stability previously determined by the method according to claim 1 using one or more conversion levels of the feedstock,
e. deducing whether or not the predetermined cut is stable.

26. A method for monitoring a mixture of hydrocarbons comprising mixing one or more hydrocarbons, the method comprises, during the mixing:
a. measuring the T₂ₘₑₐₙ/T₁ₘₑₐₙ or T₁ₘₑₐₙ/T₂ₘₑₐₙ ratio of the mixture by NMR,
b. comparing the T₂ₘₑₐₙ/T₁ₘₑₐₙ or T₁ₘₑₐₙ/T₂ₘₑₐₙ ratio determined in step a) to a threshold value of the parameter representative of the stability previously determined by means of the method according to claim 14 using mixtures of the same hydrocarbons in different proportions,
c. deducing therefrom whether or not the mixture is stable.
